(19) United States
(12) Patent Application Publication
(10) Pub. No.: US 2026/0102097 A1
(43) Pub. Date: Apr. 16, 2026

(54) CARDIAC MAPPING CATHETER

(71) Applicant: COREMAP, INC., Burlington, MA (US)

(72) Inventor: Peter S. Spector, Burlington, MA (US)

(21) Appl. No.: 19/357,755

(22) Filed: Oct. 14, 2025

Related U.S. Application Data
(60) Provisional application No. 63/707,399, filed on Oct. 15, 2024.

(51) Int. Cl. A61B 5/287; A61B 5/00; A61B 5/367

(52) U.S. Cl. CPC A61B 5/287; A61B 5/367; A61B 5/6858; A61B 5/6859; A61B 2560/0468; A61B 2562/046; A61B 2562/227

(57) ABSTRACT

The invention provides a catheter with at least one electrode pair on the head of the catheter to collect data regarding the electrical activity of the heart and analyze the data using a high-performance mapping algorithm to map the interior portions of the heart to guide catheter ablation.

### TECHNICAL FIELD

This invention relates to a cardiac mapping catheter with electrode pairs disposed thereon.

### BACKGROUND

Atrial fibrillation accounts for almost one-third of all hospital admissions for a cardiac rhythm disturbance. Atrial fibrillation, or Afib, is expressed as an uncontrolled firing of muscle fibers that results in an arrythmia. This condition, if left untreated, is associated with increased mortality due to stroke or heart failure. Atrial fibrillation may be paroxysmal or chronic and the causes are varied and often unclear. Existing treatments for Afib include medications and interventions that seek to restore and maintain normal and organized cardiac electrical activity in the atria. Medications are effective in only a small percentage of patients. As a result, clinicians often have to resort to invasive methods, including catheter ablation. Conventional ablation strategies, however, are successful only about 70% of the time. This is thought to be due to the complexities and variability of electrical activity that underlies atrial fibrillation, making the identification of the drivers and pathways of propagation of the arrhythmia difficult.

Thus, patients suffering from a conduction disorder would benefit from new methods and systems for the preventing, treating, and at least minimizing if not terminating electrical pathways in the underlying tissue which cause conduction disorders of the heart. These methods and systems would help clinicians minimize or prevent further episodes and increase the success rate of ablation treatments in patients with conduction disorders.

### SUMMARY OF THE INVENTION

The present invention provides cardiac mapping catheters comprising an elongated catheter body having a proximal portion and a distal portion. A sensing head is connected to the distal portion of the catheter body. The sensing head comprises a plurality of strut members and at least one electrode pair positioned along a strut member. The electrode pair has a first electrode curved around a first surface of the strut member and a second electrode curved around a second surface that is opposed to the first surface of the strut member.

A cardiac mapping catheter of the invention may, in a preferred embodiment, contain multiple electrode pairs positioned along the struts. Each electrode pair may also be coupled to a conductive lead. Additionally, at least one electrode from the electrode pair may be in direct contact with heart tissue while the other electrode from the electrode pair is not in direct contact with heart tissue. In preferred embodiments, the electrode pair allows for better far field effects, depth sensing, and a increased signal-to-noise ratio. Each electrode in an electrode pair may be separated by an insulating material, which may be a polymer. Optionally, the electrodes may be independent of one another. The data collected by the electrode pairs may be analyzed using a computer-based mapping algorithm. In some embodiments, strut members may extend from the elongated catheter body in a generally parallel configuration and meet at distal terminus of the sensing head. In other embodiments, strut members may independently extend from the elongated catheter body and are independently movable of one another. The strut members may meet at a proximal terminus of the sensing head and extend independently therefrom.

Additional aspects and advantages of the invention are provided in the following detailed description thereof.

### BRIEF DESCRIPTION OF DRAWINGS

FIG. 1 illustrates an exemplary cardiac mapping catheter with electrode pairs disposed thereon.

FIG. 2 illustrates an exemplary sensing head mounted on a catheter body.

FIG. 3 illustrates a second exemplary sensing head mounted on a catheter body.

FIG. 4 illustrates an exemplary side view of an electrode in contact with a strut member.

### DETAILED DESCRIPTION

The invention provides cardiac mapping catheters. Preferred catheters have an elongated body with a proximal portion and a distal portion. A sensing head is connected to the distal portion of the catheter body and comprises a plurality of strut members. At least one electrode pair is positioned along one strut member. The electrode pair has a first electrode curved around a first surface of the strut member and a second electrode curved around a second surface opposed to the first surface of the strut member.

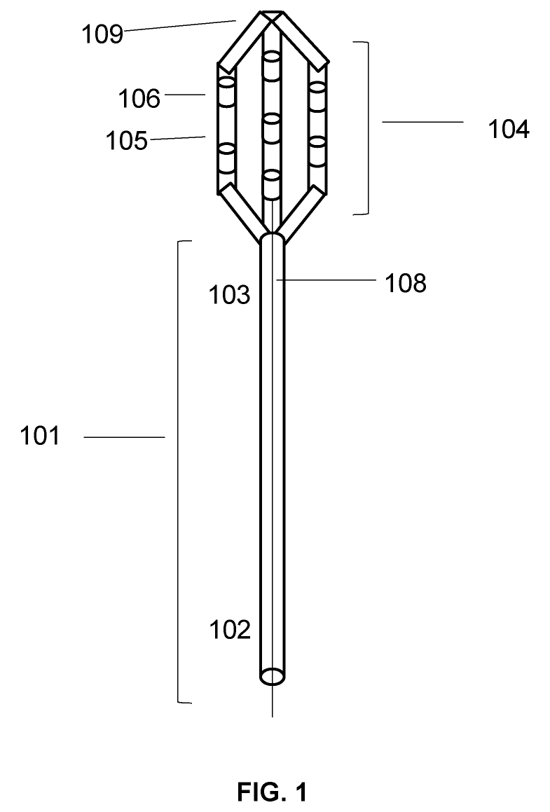

FIG. 1 shows an example of a cardiac mapping catheter of the invention. Generally, a catheter of the invention is a thin tube made from medical-grade materials. Specifically, the catheter disclosed herein is a cardiac mapping catheter to guide cardiac ablation. FIG. 1 shows a catheter comprising an elongated catheter body 101. The elongated catheter body 101 is tube-like with a hollow central passage capable of housing wires, electronics, and other internal components necessary for operation of the catheter. The hollow central passage may also be used for insertion of necessary instruments into the heart, once the catheter body has been inserted into a patient. The elongated catheter body 101 may be of varying length, thickness, and flexibility, depending on the intended application of the user. The catheter may also come in varying lengths depending on the size of the patient and point of entry into the body. For example, a catheter used for an operation on a child may be smaller than one used in an operation on an adult; or a catheter which enters the femoral vein to access the heart may be shorter than on which enters the subclavian vein to access the heart. The catheter of the present invention will be long enough to reach any interior surface of the heart from any suitable point of entry. A catheter of the present invention can also be of any suitable diameter. On average, cardiac catheters can be about 3mm in diameter or less. This may vary depending on the procedure and items which will be inside of the catheter. The catheter disclosed herein is generally flexible and capable of bending to reach any cardiac surface. The catheter body 101 may also be configured to be controlled by any suitable manual or robotic control system (not pictured).

The elongated catheter body 101 may be made of any suitable material or combination of materials including, but not limited to, natural or synthetic polymers. For example, polyamide (nylon), polyether block amide, polyurethane, polyethylene terephthalate (PET), polyimides, vinyl, silicone rubber, latex, thermoplastic elastomers, or the like. Polymers are substances or materials consisting of very large molecules called macromolecules, which are composed of many repeating subunits called monomers. Due to their broad spectrum of properties, both synthetic and natural polymers play essential and ubiquitous roles in everyday life. Polymers range from familiar synthetic plastics, such as polystyrene, to natural biopolymers such as DNA and proteins that are fundamental to biological structure and function. Polymers, both natural and synthetic, are created via polymerization of many small molecules, known as monomers. Their consequently large molecular mass, relative to small molecule compounds, produces unique physical properties including toughness, high elasticity, viscoelasticity, and a tendency to form amorphous and semicrystalline structures rather than crystals.

Polymer catheters may be further strengthened by the addition of other materials such as, stainless steel braiding, laser-cut stainless steel tubing, or other scaffold-like structures to impart desirable handling characteristics on the catheter. The catheter body 101 disclosed herein has both a proximal portion 102 and a distal portion 103.

At the end of the catheter closest to the user is the proximal portion 102. The proximal portion 102 of the catheter is where a user controls the catheter. This proximal portion 102 may be composed of the same or a different material than the other parts of the catheter body and may optionally be less flexible than the remainder of the catheter body to allow for more stability and translation of pushing force in the catheter body as a whole. The proximal portion 102 may be open at the end of the user so that a user may access any wires or controls as they exit the proximal portion 102. Data connection wires may also extend out of the open end of the proximal portion 108 to be paired with a computer or visual display. Other instruments necessary for intracardiac procedures may also be inserted into the proximal portion 102. The proximal portion 102 may optionally also include additional features to provided added grip and/or stability for the user. For example, the proximal portion 102 can optionally include a textured cover or a recess configured to allow the user to rest the proximal portion 102 on a suitable support member. The proximal portion 102 may have structural features to allow the support member to attach effectively.

The distal portion 103 of the catheter body 101 is the portion of the catheter that will be inserted into a patient. This distal portion 103 may be comprised of the same or a different material than the other parts of the catheter body. For example, the distal portion 103 may be comprised of a less rigid material than the rest of the catheter body if it is desirable to give the distal portion 103 a greater degree of flexibility. The distal portion 103 may taper down to a smaller diameter at its distal end or remain the same width as the rest of the catheter body 101. The distal portion 103 of the catheter is flexible which makes maneuvering within the heart of the patient much easier. The distal portion 103 may also be modified to equip various sensing heads 104 and attachments, as the user deems necessary. For example, the distal portion 103 may contain clips, snaps, or other recessed portions for quicker installation of various sensing heads 104 and other attachments. The distal portion 103 may also house any necessary processing units for data processing. The distal portion 103 may also house any components intended to be detected by tracking systems, such as radio-opaque markers which will be visible on X-ray imaging or sensors for use with 3D electromagnetic tracking systems.

Connected to the end of the distal portion 103 is a sensing head 104. The sensing head 104 is a portion of the catheter that comes into contact with an interior surface of the heart. The sensing head 104 is comprised of a plurality of strut members 105. In some embodiments, as is shown at FIG. 1, the strut members 105 may extend from the elongated catheter body in a generally parallel configuration. The strut members 105 may meet at a distal terminus 109 of the sensing head. The strut members 105 may be composed of any rigid or semi-rigid material including, but not limited to, polymers, metals, or some combination thereof. Based on the materials selected, the strut members may be configured to bend or remain rigid while in contact with the heart. The sensing head 104 may be any shape including, but not limited to, a paddle, one or more flexible spines, a globe shape, a basket shape, a lasso, or any other suitable shape.

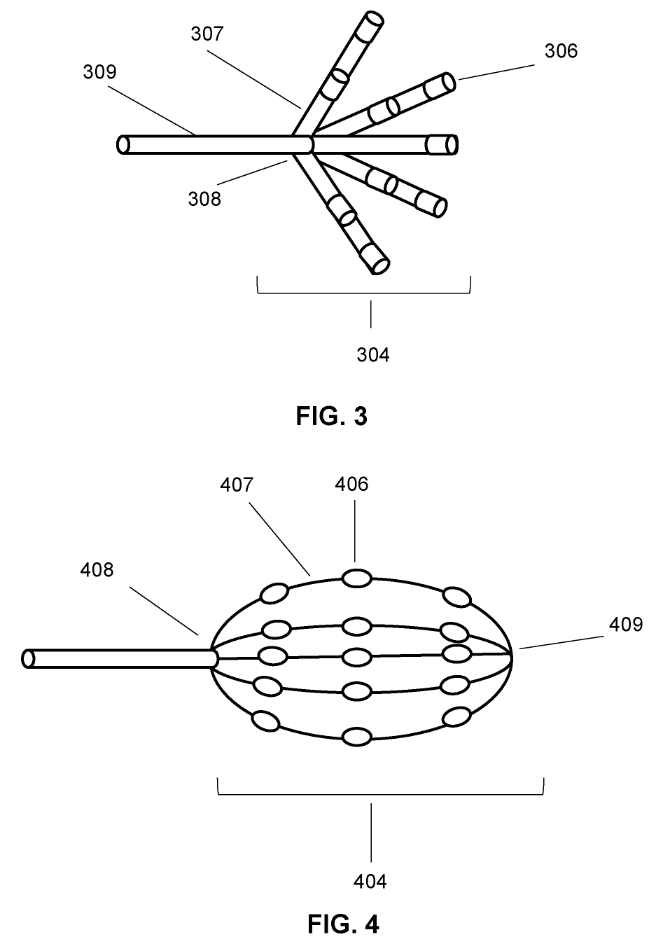

For example, FIG. 3 illustrates an exemplary sensing head 304 of a different configuration. The sensing head 304 has a plurality of struts 307 which all meet at a proximal terminus 308 and extend independently therefrom. The proximal terminus 308 is near the distal end of the elongated catheter body 309. Each strut of this alternate configuration contains one or more electrode pair(s) 306.

FIG. 4 also illustrates and exemplary sensing head 404 of an alternate configuration. The sensing head 404 has a plurality of electrode pairs 406 disposed on each strut 407. The struts 407 meet at both a proximal terminus 408 and a distal terminus 409 to form a generally round sensing head 404.

The sensing head 104 may also have one or more electrode pairs 106 positioned along one or more strut members 105. The electrodes 106 are configured for detection and transmission of the heart's electrical activity through successive cardiac cycles.

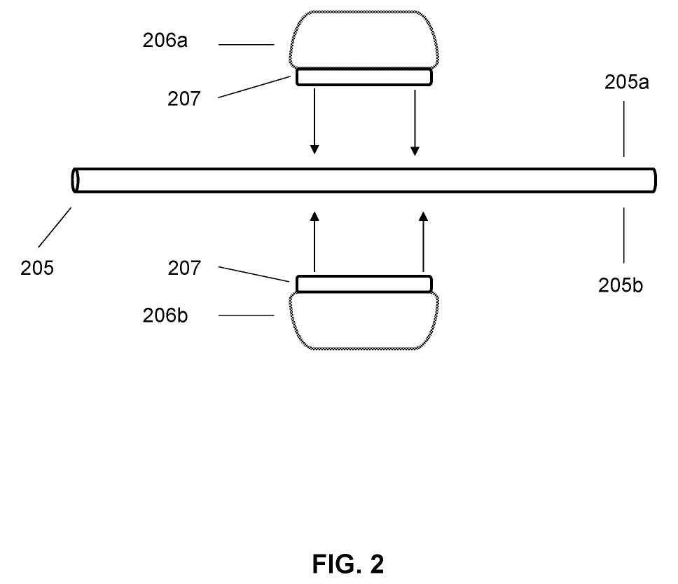

The electrodes in the electrode pair 106 are typically made of a metal such as gold, stainless steel, or any other suitable metal and may be coated with other materials to decrease impedance, improve biocompatibility, increase surface area, protect the electrode from oxidation, or increase signal-to-noise ratio (see FIG. 2 - 207). The insulation material 207 can be an electrical insulator. An electrical insulator is a material in which electric current does not flow freely. The atoms of the insulator have tightly bound electrons which cannot readily move. Other materials—semiconductors and conductors—conduct electric current more easily. The property that distinguishes an insulator is its resistivity; insulators have higher resistivity than semiconductors or conductors. The most common examples are non-metals. For example, polymers, glass, porcelain, ceramic, rubber, or other plastics can be used as the insulation material.

Individual electrodes or electrodes disposed in pairs may vary in shape, size, sensitivity, and orientation – meaning, the electrodes 106 may be mounted on the head 104 so that any portion of the electrode (top, side, etc.) is touching the interior portion of the heart. The electrodes 106 may be any suitable shape, dependent on their intended application. Spacing of the electrodes 106 may also vary dependent on their intended application.

In some embodiments, there may be multiple electrode pairs positioned on each strut. As shown in FIG. 2, the electrode pairs 106 may be comprised of two separate, curved electrodes (see 206a and 206b). The first electrode 206a may be curved around a first surface 205a of the strut member 205 and the second electrode 206b may be curved around a second surface 205b of the strut member 205, wherein the second surface is opposed to the first surface. Optionally, in the one or more electrode pairs, each electrode 206a and 206b may be separated by an insulating material 207.

The electrodes 106 may be configured so that at least one electrode from the electrode pair is in direct contact with heart tissue and the other electrode from the electrode pair is not in direct contact with heart tissue. In the present example at FIG. 2, the first electrode 206a will not be in direct contact with the heart but, the second electrode 206b will be. Placement of the electrode pair in this fashion allows for signal-to-noise improvement by utilizing the electrodes as dipole pairs, better distinction of far field effects, depth sensing, and a increased signal-to-noise ratio.

Better distinction of far field effects, depth sensing, and increased signal-to-noise ratio are important in the context of the present invention. Due to the nature of cardiac conduction and heart function, it is necessary to differentiate between multiple signals from multiple locations and throughout multiple conduction layers.

The cycles of the heart are determined by The Cardiac Conduction System (CCS, also called the conduction system of the heart), which is the main driver of proper heart function. The CCS transmits the signals generated by the sinoatrial node – the heart's pacemaker, to cause the heart muscle to contract, and pump blood through the body's circulatory system. The pacemaking signal travels through the right atrium to the atrioventricular node, along the bundle of His, and through the bundle branches to Purkinje fibers in the walls of the ventricles. The Purkinje fibers transmit the signals more rapidly to stimulate contraction of the ventricles. The conduction system consists of specialized heart muscle cells, situated within the myocardium. There is a skeleton of fibrous tissue that surrounds the conduction system. Dysfunction of the conduction system can cause irregular heart rhythms including rhythms that are too fast, too slow, or chaotic and asynchronous.

Electrical signals arising in the SA node (located in the right atrium) stimulate the atria to contract. Then the signals travel to the atrioventricular node (AV node), which is located in the interatrial septum. After a short delay that gives the ventricles time to fill with blood, the electrical signal diverges and is conducted through the left and right bundle branches of His to the respective Purkinje fibers for each side of the heart, as well as to the endocardium at the apex of the heart, then finally to the ventricular epicardium; causing the ventricles to contract. These signals are generated rhythmically, which results in the coordinated rhythmic contraction and relaxation of the heart.

On the microscopic level, the wave of depolarization propagates to adjacent cells via gap junctions located on the intercalated disc. The heart is a functional syncytium as opposed to a skeletal muscle syncytium. In a functional syncytium, electrical impulses propagate freely between cells in every direction, so that the myocardium functions as a single contractile unit. This property allows rapid, synchronous depolarization of the myocardium. While advantageous under normal circumstances, this property can be detrimental, as it has potential to allow the propagation of incorrect electrical signals.

The electrode pair 106 of the present invention helps to differentiate between near field and far field signals. Near field signals are types of cardiac activations that occur close to the electrode pair. For example, if electrode 206b in the electrode pair is touching the endocardium (the thin layer that lines the interior portion of the heart), electrical activations nearby in the myocardium (the muscle of the heart that underlies the endocardium) can be easily detected as a near field signal. The signal will likely be very strong since it is closer to the electrode. Although the signal would be detected by both electrodes in the pair, it will be received as a stronger signal in the electrode in contact with the tissue 206b than the electrode not touching the tissue 206a. This difference in signal strength can be used to determine the precise location of the activation signal.

The first electrode 206a that is not in contact with the tissue helps to provide better far field effects. Far field signals are those which originate further from the electrode but, are still detected (oftentimes, as a much weaker signal). Far field signals can be different than the ones that appear in the near field or can be the same signal originating from a further point and traveling towards the electrode. Far field signals exist because the heart is capable of multiple layers of conduction and performs electrical conduction in different portions of the heart, all which are close by. Conduction occurring in another nearby chamber of the heart that is picked up by an electrode in a different chamber could also be interpreted as a far field effect.

By way of example, a signal that originates far away from the electrode pair 106 and travels towards it may be detected as a far field signal and a near field signal (by both the first electrode 206a and the second electrode 206b). However, a signal that occurs in a different part of the heart may only be picked up as a far field signal by the first electrode 206a and could likely be ignored. Because of the nature of the electrode pair 106, the catheter disclosed herein is not disrupted by far field signals and interprets electrical signal conduction with much greater accuracy.

The electrode pair 106 also provides better depth sensing. Due to the structure of the heart and the nature of cardiac conduction (outlined above), conduction can occur in multiple layers of the heart. The use of a first electrode 206a and a second electrode 206b provides multiple readings of each electrical signal that occurs in the heart. For example, conduction which occurs closer to the electrode pair 106 will be interpreted as a stronger signal by both the first electrode 206a and the second electrode 206b. This allows for a much more accurate determination of the precise location of an electrical impulse that is detected by the electrode pair 106.

The configuration of the electrode pair 106 also provides an increased signal-to-noise ratio. Signal-to-noise ratio is a measure that compares the level of a desired signal to the level of background noise. In the present invention, the signal to be detected is an electrical impulse of the heart, typically one that leads to normal or abnormal conduction. Background noise can be any undesired signal that is picked up by the electrode pair 106.

Signal-to-noise is an important parameter that affects the performance and quality of systems that process or transmit signals. A high signal-to-noise means that the signal is clear and easy to detect or interpret, while a low signal-to-noise means that the signal is corrupted or obscured by noise and may be difficult to distinguish or recover. Signal-to-noise can be improved by various methods, such as increasing the signal strength, reducing the noise level, filtering out unwanted noise, or using error correction techniques. Signal-to-noise can be calculated using different formulas depending on how the signal and noise are measured and defined. The most common way to express signal-to-noise is in decibels, which is a logarithmic scale that makes it easier to compare large or small values. Other definitions of signal-to-noise may use different factors or bases for the logarithm, depending on the context and application. Once determined, signal-to-noise may be reduced to a pre-determined, constant value. Signal-to-noise may be calculated by a computer system paired to the catheter.

The use of a first electrode 206a and a second electrode 206b can provide and increased signal-to-noise ratio by utilizing the input from both electrodes to determine if a signal is background noise or a signal to be detected by the electrode pair 106. For example, if an electrical impulse from a different part of the heart is detected as a weak signal by both the first electrode 206a and the second electrode 206b, it can likely be interpreted as background noise and discounted from the mapping determination. By filtering out unwanted and unnecessary noise, a cleaner and more reliable signal is achieved and thus, a more accurate map of the heart is provided. Or, if the electrode 206a is in contact with the cardiac tissue and 206b is in contact with the blood in the heart cavity, the signal on electrode 206a will be a combination of the directly-sensed cardiac signal and any noise induced in the wires and connections leading up to the electrode. The signal on 206b will by contrast primarily be the same noise seen by 206a. Thus, selective amplification of only the difference between the two electrodes can dramatically increase the signal to noise ratio.

Each electrode pair may be connected to a conductive lead 108. The conductive lead 108 is an electrical connector consisting of a length of wire or other suitable material that is designed to connect two locations electrically. Conductive leads 108 of the present invention extend from the electrode pair 106 and through the catheter body 101 and out of the proximal portion 102. The conductive lead(s) 108 are responsible for transmitting data received by the electrodes to a computer or processing unit (not pictured). The conductive lead(s) 108 may connect to a computer, a processing chip or device, a visual display, or any other apparatus capable of receiving and transmitting electrical input signals. The conductive leads 108 may comprise wires, cables, cords, or any other suitable device for transmitting electrical signals.

Once data is collected by the electrode pair 106 it may be analyzed using a high-performance mapping algorithm. In the context of the present invention, an algorithm is a finite sequence of rigorous instructions, typically used to perform a computation. Algorithms are used as specifications for performing calculations and data processing.

The present invention is configured for use with a high performance mapping algorithm. High performance mapping algorithms are capable of receiving and transmitting large quantities of data into a useable form. For example, the cardiac mapping catheter of the present invention is capable of receiving a large quantity of data from the electrode pair(s) 106 and transforming it into a visual representation of the heart and its conduction patterns.

Optionally, the algorithm may be in the form of a computer program. A computer program is a sequence or set of instructions in a programming language for a computer to execute. It is one component of software, which also includes documentation and other intangible components. A computer program in its human-readable form is called source code. Source code needs another computer program to execute because computers can only execute their native machine instructions. Therefore, source code may be translated to machine instructions using a compiler written for the language. The resulting file is called an executable.

The cardiac mapping catheter of the present invention is also capable of utilizing high performance computing (HPC). HPC uses supercomputers and computer clusters to perform advanced computing tasks and solve advanced computation problems. HPC integrates systems administration (including network and security knowledge) and parallel programming into a multidisciplinary field that combines digital electronics, computer architecture, system software, programming languages, algorithms and computational techniques. HPC technologies are the tools and systems used to implement and create high performance computing systems. Recently, HPC systems have shifted from supercomputing to computing clusters and grids. This may be applicable because it is within the scope of the present invention to tie multiple computers together to perform the necessary mapping functions. Additionally, the computer or multiple computers may tie into various hospital and other network systems for tracking data related to the procedure, tracking patient data, security, and for patient recordkeeping.

### Incorporation by Reference

References and citations to other documents, such as patents, patent applications, patent publications, journals, books, papers, web contents, publicly accessible databases, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

WHAT IS CLAIMED IS:

1. A cardiac mapping catheter comprising: an elongated catheter body having a proximal portion and a distal portion; a sensing head connected to the distal portion of the catheter body, the sensing head comprising a plurality of strut members; and at least one electrode pair positioned along at least one strut member, wherein the electrode pair has a first electrode curved around a first surface of the strut member and a second electrode curved around a second surface opposed to the first surface of the strut member.

2. The cardiac mapping catheter of claim 1, wherein a multiple of electrode pairs are positioned along the struts.

3. The cardiac mapping catheter of claim 1, wherein strut members extend from the elongated catheter body in a generally parallel configuration and meet at distal terminus of the sensing head.

4. The cardiac mapping catheter of claim 1, wherein strut members independently extend from the elongated catheter body and are independently movable with respect to one another.

5. The cardiac mapping catheter of claim 4, wherein the strut members meet at a proximal terminus of the sensing head and extend independently therefrom.

6. The cardiac mapping catheter of claim 1, wherein each electrode pair is coupled to a conductive lead.

7. The cardiac mapping catheter of claim 1, wherein at least one electrode from the electrode pair is in direct contact with heart tissue and the other electrode from the electrode pair is not in direct contact with heart tissue.

8. The cardiac mapping catheter of claim 7, wherein the electrode pair allows for signal-to-noise improvement by utilizing the electrodes as dipole pairs, better distinction of far field effects, depth sensing, or increased signal-to-noise ratio.

9. The cardiac mapping catheter of claim 1, wherein the data collected by the at least one electrode pair are analyzed using a high-performance mapping algorithm.

10. The cardiac mapping catheter of claim 1, wherein each electrode in the at least one electrode pair are separated by an insulating material

11. The cardiac mapping catheter of claim 10, wherein the insulating material is a polymer.

12. The cardiac mapping catheter of claim 1, wherein the electrodes are independent of one another.
